(19) United States
(12) Patent Application Publication
(10) Pub. No.: US 2019/0046556 A1
(43) Pub. Date: Feb. 14, 2019

(54) Methods and Pharmaceutical Compositions for the Prophylactic Treatment of Peritoneal Carcinomatosis

(71) Applicants: INSERM (Institut National de la Sante et de la Recherche Medicale), Paris (FR); Universite Paris Diderot - Paris 7, Paris (FR); Assistance Publique-Hopitaux de Paris (APHP), Paris (FR)

(72) Inventor: Marc POCARD, Paris (FR)

(21) Appl. No.: 16/074,598

(22) PCT Filed: Jan. 27, 2017
(86) PCT No.: PCT/EP2017/051802

§ 371 Date: Aug. 1, 2018

(30) Foreign Application Priority Data
Feb. 4, 2016 (EP) 16305126.1

(51) Int. Cl. A61K 31/715; A61K 45/06; A61P 35/04

(52) U.S. Cl. CPC A61K 31/715; A61K 45/06; A61K 31/282; A61K 2300/00; A61P 35/04

(57) ABSTRACT

The present invention relates to methods and pharmaceutical compositions for the prophylactic treatment of peritoneal carcinomatosis. In particular, the present invention relates to a method for the prophylactic treatment of peritoneal carcinomatosis in a patient in need thereof comprising administering to the patient a therapeutically effective combination of at least one dextrin polysaccharide and at least one chemotherapeutic agent.

### FIELD OF THE INVENTION

The present invention relates to methods and pharmaceutical compositions for the prophylactic treatment of peritoneal carcinomatosis.

### BACKGROUND OF THE INVENTION

Peritoneal carcinomatosis (PC) is a fatal form of metastasis that occurs when intraabdominal cancers invade into the peritoneal cavity and attach to the peritoneum, a resilient tissue lining the abdominal cavity and its internal organs. Peritoneal carcinomatosis can also occur following surgical resections of intra-abdominal cancers, which releases cancer cells, blood, and lymph into the peritoneal cavity. For instance, peritoneal carcinomatosis is the second most frequent site of recurrence in colorectal cancer, involving 25 to 35% of the recurrence (1-3). Metachronous PC will occur in 2 to 19% of the patients with colorectal cancer (4). This evolution is associated to a bad prognosis. Median of survival without any treatment is about 5 months (5). Nowadays, with the systemic chemotherapy, the complete resection of the PC and the intraperitoneal chemotherapy, the 5-years overall survival is estimated between 45 and 51% with a median of survival at 41 months (6-8). The risk factors of metachronous PC are well known, T4 stage, perforated tumour, positive cytology or synchronous ovarian metastasis (4). In case of ovarian metastasis, the incidence of PC is estimated between 56 to 62% for colon cancer primary tumor (9,10). In such risk situations, the standard therapeutic attitude is not defined and improved and more effective pharmaceutical composition and methods for the prophylactic treatment of peritoneal carcinomatosis are thus needed.

The nodule of PC are made up with severals types of cells, tumoral cells and stroma cells, including macrophages, lymphocytes, endothelial cells, and a particular type of fibroblast, the CAF (carcinoma associated fibroblast) (11). The CAF come from bone marrow stem cells, from peritoneal fibroblast or from mesothelials cells via the mesothelial-to-mesenchymal transition (MMT) (12,13). The interaction between CAF and tumoral cells contributes to tumor progression. Indeed, the tumoral cells are activating by the CAF, it results in increasing tumoral agressivity, metastatic potentiel, chemoresistance. The CAF are also activated and secrete growth factor, pro angiogenic factor (14-17).

Icodextrine (ICDX) 4% is an glucose polymer linked by an alpha-1,4-glucosidic bond. It's a colloid solution which is slowly absorb in peritoneal cavity through lymphatic system. This high molecular weight molecule is degraded by the alpha-amylase which exists in the serum but not in the peritoneum. It stays in the peritoneum between 3 to 5 days (18). This solution is used to reduce post operative adhesion. The mechanism of action involves the phenomenon of hydroflotation which separates the different tissues during the early post operative phase which is the critical phase of adhesions formation (19). This solution could also inhibits the MMT. In a rat model, Klink et al. found that ratio between mesothelial cells and mesenchymal cells was higher when the rat were treated by ICDX (20).

### SUMMARY OF THE INVENTION

The present invention relates to methods and pharmaceutical compositions for the prophylactic treatment of peritoneal carcinomatosis. In particular, the present invention is defined by the claims.

### DETAILED DESCRIPTION OF THE INVENTION

There is no standard treatment in patients with risks of peritoneal carcinomatosis. Icodextrin 4% (ICDX), presently used to prevent postoperative abdominal adhesions, could inhibit the coactivation of the tumoral cells and the microenvironment cells which is essential to the development of the peritoneal carcinomatosis. The aim of the inventors was to investigate the inhibition of peritoneal carcinomatosis with ICDX and chemotherapy. The inventors created a model of growing PC in mice by injecting IP tumoral cells of colonic cancer CT26. Cells and treatments were injected simultaneously, to mimic clinical situation as peroperative surgical resection of a colon cancer with free tumor cells in the peritoneum. Five groups were done: CT26 (control group), CT26+ICDX (ICDX group), CT26+chemotherapy (oxaliplatin and 5FU) (chemo group), CT26+chemotherapy+ICDX (ICDX chemo group), ICDX (toxicity group). Killing of animals was on day 15. PC was evaluated with peritoneal cancer index (PCI) adapted to rodents. Means of PCI were compared with a non parametric Wilcoxon test. In the chemo group, PCI was significantly lower than in the control group (3,2 versus 8,4, p=0.02). ICDX had a synergetic effect on PC with chemotherapy, indeed PCI in ICDX chemo group was lower than in chemo group (1,4 versus 3,2, p=0.04). There were no difference between control group and ICDX group. There was no morbidity linked to ICDX in toxicity group. Thus a pharmaceutical composition comprising a therapeutically effective combination of at least one dextrin polysaccharide and at least one chemotherapeutic agent would be suitable for the prophylactic treatment of peritoneal carcinomatosis at the end of a curative surgery for cancer.

Accordingly a first object of the present invention relates to a method for the prophylactic treatment of peritoneal carcinomatosis in a patient in need thereof comprising administering to the patient a therapeutically effective combination of at least one dextrin polysaccharide and at least one chemotherapeutic agent.

As used herein, the term “prophylactic treatment” refers to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease. It is also to be appreciated that the various modes of treatment or prevention of medical conditions as described are intended to mean “substantial,” which includes total but also less than total treatment or prevention, and wherein some biologically or medically relevant result is achieved.

As used herein, the term “peritoneal carcinomatosis,” refers to the neoplastic involvement of the peritoneum, typically seen as wide-spread seeding or growth of tumor masses or metastases. Peritoneal carcinomatosis can result from primary or secondary carcinomas. Primary peritoneal carcinomas arise from peritoneum cells and since the mesothelium of the peritoneum and the germinal epithelium of the ovary have the same embryologic origin, the peritoneum retains the multipotentiality allowing for the development of a primary carcinoma that can then spread within the peritoneal cavity. Primary carcinomas that cause peritoneal carcinomatosis and are contemplated for treatment using the disclosed methods and agents include malignant mesothelioma, benign papillary mesothelioma, desmoplastic small round cell tumors, peritoneal angiosarcoma, leiomyomatosis peritonealis disseminata (LPD), and peritoneal hemangiomatosis. Additionally, ovarian cancer arising in women after bilateral oophorectomy is included as a primary peritoneal cancer that can result in peritoneal catcinomatosis. Much more commonly, peritoneal carcinomatosis results from a cancer that arises in an anatonomically separate location and later metastasizes to the peritoneal cavity. Numerous cancers can produce peritoneal carcinomatosis including cancers of the endometrium, fallopian tubes, ovaries, uterus, colon, rectum, small bowel, gall bladder, bile duct, appendix, stomach, pancreas, liver and breast.

In some embodiments, the peritoneal carcinomatosis results from ovarian cancer. As used herein, “ovarian cancer” or “ovarian tumor” includes any tumor, cell mass or micrometastasis derived from, or originating from cells of the ovary. This includes tumors originating from the epithelial cell layer (serous) of the ovary. Ovarian cancer further includes secondary cancers of ovarian origin and further includes recurrent or refractory disease.

In some embodiments, the peritoneal carcinomatosis is pseudomyxoma peritonei, the peritoneal dissemination of an appendiceal mucinous epithelial neoplasm, a relatively slow growing cancer that is characterized by the excessive production of mucinous ascites. (Smeenk R M, et al. Pseudomyxoma peritonei. Cancer Treat Rev 2007, 33:138-145).

Accordingly, the present invention provides a method of reducing the risk of peritoneal carcinomatosis in a patient that has had an intra-abdominal cancer removed, said method comprising administering to said patient a therapeutically effective combination of at least one dextrin polysaccharide and at least one chemotherapeutic agent.

In some embodiments, the intra-abdominal cancer is located at or near the colon, at or near the ovary, at or near the rectum, at or near the stomach, or at or near the pancreas of the patient. In some embodiments, the patient suffers from a metastatic cancer (i.e. stage IV according to the TNM classification).

As used herein, the term “dextrin polysaccharide” means a glucose polymer which is produced by the hydrolysis of starch and which consists of glucose units linked together by means mainly of α-1,4 linkages. Typically dextrins are produced by the hydrolysis of starch obtained from various natural products such as wheat, rice, maize and tapioca. In addition to α-1,4 linkages, there may be a proportion of α-1,6 linkages in a particular dextrin, the amount depending on the starch starting material.

In some embodiments, the dextrin polysaccharide is in the form of either unsubstituted dextrin (as obtained by the hydrolysis of starch) or may be substituted by one or more different groups. The substituents may be negatively charged groups, for instance, sulfate groups, neutral groups, or positively charged groups, for instance, quaternary ammonium groups. In the case where the substituent group is sulfate, the sulfated polysaccharide typically contains at least one sulfate group per saccharide (glucose) unit.

Any dextrin is a mixture of polyglucose molecules of different chain lengths. As a result no single number can adequately characterize the molecular weight of such a polymer. Accordingly, various averages are used, the most common being the weight average molecular weight (Mw) and the number average molecular weight (Mn). Mw is particularly sensitive to changes in the high molecular weight content of a polymer whilst Mn is largely influenced by changes in the low molecular weight content of the polymer. In some embodiments the Mn of the dextrin is in the range of from 1,000 to 30,000. In some embodiments, the Mn is from 3,000 to 8,000 and the Mw is from 5,000 to 50,000.

The dextrin polysaccharide used in the present invention is typically water soluble or at least forms a suspension in water or a gel formulation.

In some embodiments, the dextin polysaccharide is icodextrin. Icodextrin is a starch-derived, branched, water-soluble glucose polymer linked by α-(1→4) and less than 10% α-(1→6) glycosidic bonds, making it a type of dextrin. Its Mw is between 13,000 and 19,000 Daltons and its Mn between 5,000 and 6,500 Daltons. The substance is a white to off-white solid, and the solution is clear and colourless to pale yellow. Icodextrin (INN, USAN) is used in form of an aqueous solution for peritoneal dialysis under the trade name Extraneal®, and after gynecological laparoscopic surgery for the reduction of post-surgical adhesions (fibrous bands that form between tissues and organs) under the trade name Adept®.

As used herein, the term “chemotherapeutic agent” refers to any compound that can be used in the treatment, management or amelioration of cancer, including peritoneal carcinomatosis, or the amelioration or relief of one or more symptoms of a cancer. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomycins, actinomycin, authramycin, azascrine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycins, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′ 2″-trichlorotriethylamine; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; chlorambucil; gemcitabine; 6-thioguanine; mercaptopurine; methotrexate; platinum analogs such as cisplatin and carboplatin; etoposide (VP-16); ifosfamide; mitomycin C; mitoxantrone; vinblastine; vincristine; vinorelbine; navelbine; novantrone; teniposide; daunomycin; aminopterin; xeloda; ibandronate; difluoromethylornithine (DMFO); retinoic acid; esperamicins; capecitabine; imexon; tyrosine kinase inhibitors, such as epidermal growth factor receptor tyrosine kinase inhibitor erlotinib; and pharmaceutically acceptable salts, acids or derivatives of any of the above.

In some embodiments, the chemotherapeutic agent is capecitabine, carboplatin, cisplatin, cyclophosphamide, docetaxel, doxorubicin, epirubicin, erlotinib, 5-fluorouracil, gemcitabine, irinotecan, leucovorin, oxaliplatin, paclitaxel or topotecan.

As used herein, the term “therapeutically effective combination” as used herein refers to an amount or dose of the dextrin polysaccharide together with the amount or dose of the chemotherapeutic agent that is sufficient to prevent peritoneal carcinomatosis. The amount of the dextrin polysaccharide and the chemotherapeutic agent in a given therapeutically effective combination may be different for different individuals and different tumor types, and will be dependent upon the one or more additional agents or treatments included in the combination. The “therapeutically effective amount” is determined using procedures routinely employed by those of skill in the art such that an “improved therapeutic outcome” results. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein the term “co-administering” as used herein means a process whereby the combination of the dextrin polysaccharide and the chemotherapeutic agent, is administered to the same subject. The dextrin polysaccharide and the chemotherapeutic agent may be administered simultaneously, at essentially the same time, or sequentially. The dextrin polysaccharide and the chemotherapeutic agent need not be administered by means of the same vehicle. The dextrin polysaccharide and the chemotherapeutic agent may be administered one or more times and the number of administrations of each component of the combination may be the same or different. In addition, the dextrin polysaccharide and the chemotherapeutic agent need not be administered at the same site.

In some embodiments, the dextrin polysaccharide and the chemotherapeutic agent are administered to the patient in the same pharmaceutical composition.

Thus the present invention also provides a pharmaceutical composition comprising an amount of at least one dextrin polysaccharide and at least one chemotherapeutic agent.

Typically, the pharmaceutical composition of the present invention is allowed to remain in the body cavity over the period during which the risk for tumoral dissemination is maximal. Typically, the composition should remain in the body cavity for a period of up to 7 to 15 days after the tumor resection (i.e. surgery). Typically, the pharmaceutical composition of the present invention is applied to the body cavity in a volume in the range 500-2000 ml and, more preferably, about 1000 ml-1500 ml. It will be appreciated that the concentration of the pharmaceutical composition of the invention, the timing of administration and the dwell time are variable and may be selected according to a user's requirements.

According to the invention, the pharmaceutical composition is suitable for intraperitoneal administration. In some embodiments, the pharmaceutical composition of the present invention is an aqueous solution or suspension or a gel formulation. Typically, the pharmaceutical composition of the present invention is applied using conventional techniques. Coating, dipping, spraying, spreading and solvent casting are possible approaches. More particularly, said applying is manual applying, applicator applying, instrument applying, manual spray applying, aerosol spray applying, syringe applying, airless tip applying, gas-assist tip applying, catheter applying, endoscopic applying, arthroscopic applying, encapsulation scaffold applying, image-guided applying, radiologic applying, brush applying, wrap applying, or drip applying.

Typically, the pharmaceutical composition of the present invention typically includes additional pharmaceutically acceptable excipients, and optionally sustained-release matrices, such as biodegradable polymers. As used herein the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. For instance, any one or more of the following elements can be added to the pharmaceutical composition of the present invention: a suitable lubricant such as a phosphospholipid; a calcium binding agent such as EDTA or sodium citrate; a hyaluronate; a prostacyclin or an analogue thereof, a glycocosolaminoglycan; an antibiotic agent or a material/agent which is associated with preventing an infection or build up of bacteria or foreign bodies or the like. The pharmaceutical composition of the present invention may also include a fibrinolytic agent or an analogue thereof, an anti-inflammatory agent or an analogue thereof, and/or colorant (e.g. methylene blue). Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts). Dispersions can also be prepared in glycerol, liquid polyethylene glycols, and mixtures thereof and in oils. Under ordinary conditions of storage and use, these preparations contain a preservative to prevent the growth of microorganisms. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. The pharmaceutical composition of the invention is typically prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

The present invention also relates to a kit for performing the method of the present invention, wherein said kit comprises the pharmaceutical composition of the present invention. In some embodiments, the kit comprises means for distributing the pharmaceutical composition on the surface of the tissue where the tumor was resected (dripper, spray, vacuum, pipette or sealed pipette, patches, dressing, elastoplasts band-aid or brush for example).

The invention will be further illustrated by the following figures and examples. However, these examples and figures should not be interpreted in any way as limiting the scope of the present invention.

### FIGURES

FIG. 1 shows the MTT assay.

FIG. 2 shows the Cell migration assay.

FIG. 3 shows the PCI comparison.

FIG. 4 shows the number of areas involved.

FIG. 5 shows PCI at 21 days from injection of OVCAR cells in nude mice, regarding 3 different treatments tested.

### EXAMPLE 1

Material & Methods

Cell Line and Reagents.

Murin tumoral cells of a colonic cancer transfected by luciferase, CT26 LUC, were given by Professor Lea Eisembac (Weizmann Institute of science Rehovot, Israel). CT26 LUC were maintained in DMEM medium supplemented with 10% fetal bovine serum, 2 mM L-Glutamine and antibiotics. They were grown at 37° C. in a humidified incubator with 5% CO₂. Chemotherapy and ICDX were given by the department of general and visceral surgery of Lariboisière Hospital.

Viability Assay.

To measure cell viability and growth, MTT assay (3-[4,5-dimethylthiazol-2-yl]-2,5 diphenyl tetrazolium bromide) was done. Cells, seeded in 96-well plates (2×10⁴ cells/well), were cultured in complete DMEM medium for 24 h. Cells were then treated with different concentrations of ICDX (0%, 30%, 50%, 70% and 90%) for 24, 48 and 72 h. The cells, after treatment were incubated with MTT for 3 h. The yellow tetrazolium salt (MTT) is reduced in metabolically active cells to form insoluble purple formazan crystals, which are solubilized by the addition of DMSO. The color was then quantified by spectrophotometric measurement at 570 nm wavelength on a microtiter plate reader.

Adhesion Assay.

Cells were seeded at a density of 6.10⁴ cells/well in 24-well plates, previously prepared with 0.2% filtered gelatin, on a block of ice with 500 μL of medium or 250 μL of medium and 250 μL of ICDX. Plates were put in the incubator. At the different time (0, 5, 10, 15, 20, 25, 30, 60, 90, 120 minutes), the different well were washed with PBS. The wells were then colored with eosin and blue colorant. In each well, we did 4 counting of the cells which were adherent.

Cell Migration Assay.

Cells were seeded at a density of 5.10⁵ cell/well in 6-well plates and incubated for 3 days with complete medium. When cells were 70-80% confluent, we made an “injury” in the well. Medium were modified (complete medium as the control well, ICDX 50% as ICDX well, medium and oxaliplatine 0.039 ng/mL as chemo well, medium and oxaliplatine and ICDX as ICDX chemo well). Pictures of the invasion front were taken at different time and rate of migration was calculated.

In vivo experimentation. Female mice BALB/C were used. They received an intraperitoneal injection of CT26 LUC associated with the treatment. The follow up of the PC was done with bioluminescence imaging. Once a week, mice were injected with luciferin in the peritoneum (10 μL/g). Ten minutes after, the mouse was put under the bioluminescence camera for 10, 5 and 2 minutes depending on the time of the experimentation. Twice a week the well-being of the mouse was checked by analysing signs of pain, changing in behaviour, loss of weight, dehydration signs. At day 15, the mice were killed by cervical dislocation. A laparotomy was performed by doing a xypho-pubic incision. We used the peritoneal carcinomatosis index (PCI) for the rodent to describe PC, as prior published (data not shown) (21).

Severals samples were taken to make histological analysis.

Five groupes of mice were done:

Control group: CT26 LUC (5.10³ cells)

ICDX group: CT26 LUC and ICDX (20 mL/kg)

Chemo group: CT26 LUC and Chemotherapy (Oxaliplatine 6 mg/kg and 5 fluorouracil 30 mg/kg)

ICDX Chemo group: CT26 LUC and ICDX and Chemotherapy

Toxicity group: ICDX

Statistic Analysis

Discrete and continuous variables were expressed by mean (standard deviation). Means were comparated with a Mann-Withney non parametric test. To compare more than 2 groups, Kruskal-Wallis test was used. We used p<0.05 as a significantly value.

Results

Viability Assay.

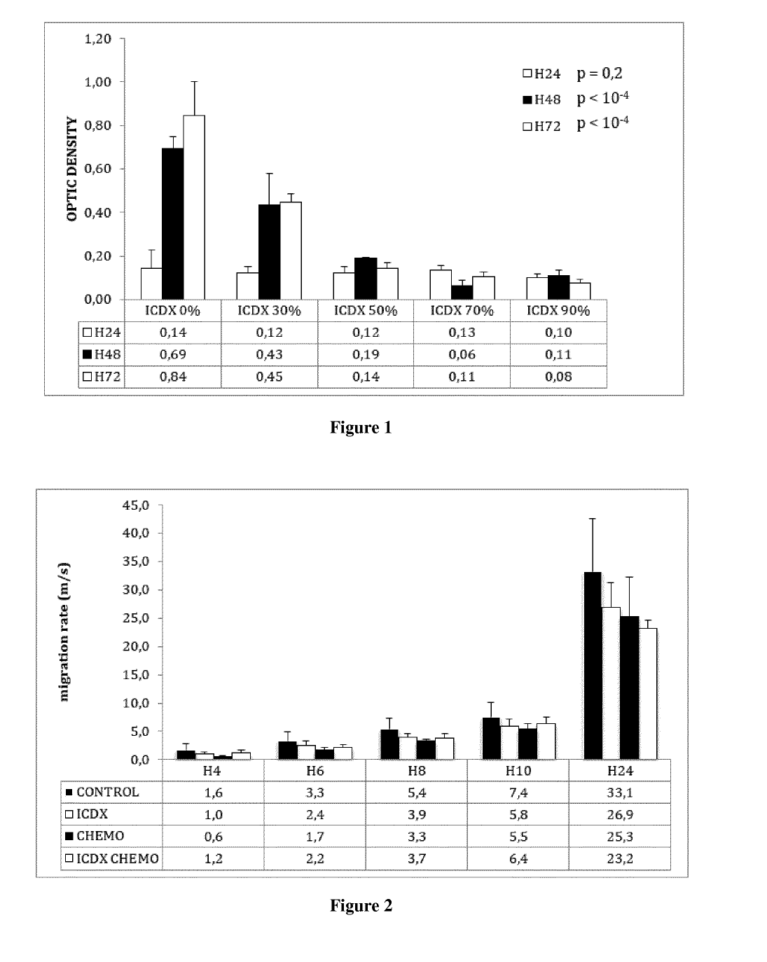

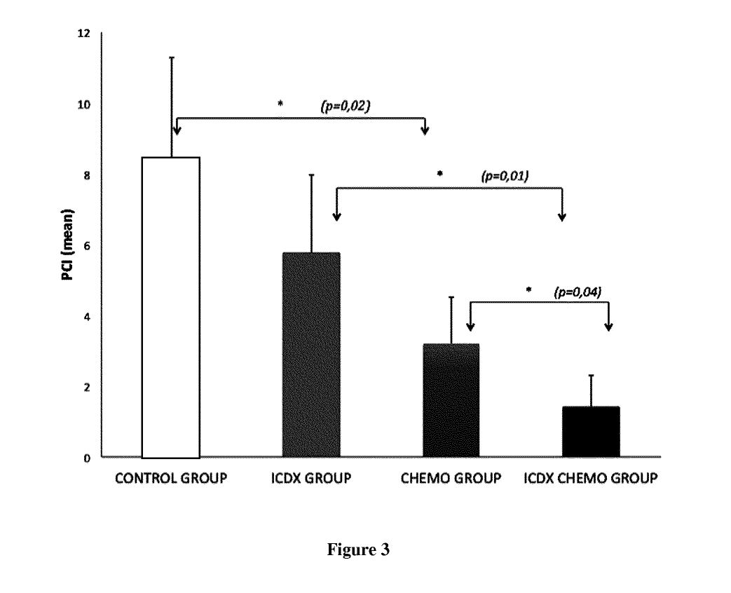

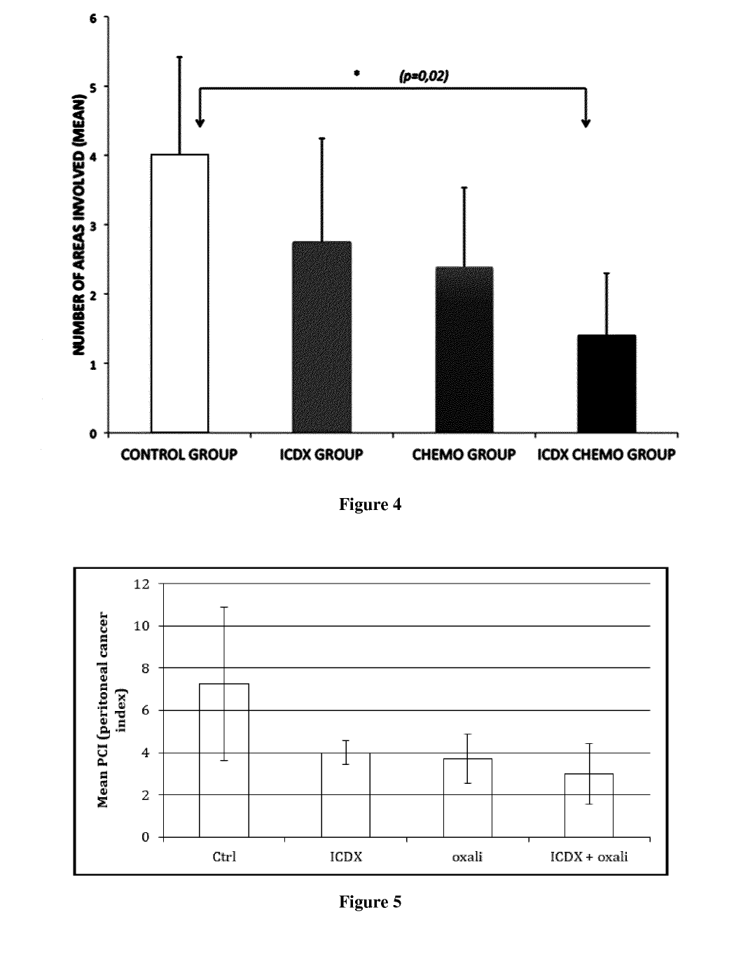

Viability of cells was significantly lower when ICDX was present in the medium at H48 and H72 (FIG. 1). We compared at day 2 and 3 ICDX 0% versus ICDX 50%. Mean optic densities were lower with ICDX 50% at day 2 (0.19 versus 0.60, p<10⁻⁴) and day 3 (0.12 versus 0.64, p<10⁻⁴).

Adhesion Assay.

No cells were adherent before 15 minutes. At t=15 minutes, there were significantly more cells in the control well than in the ICDX well (4 versus 0, p=0.03). At t=20 minutes and after the mean were not different (Table 1).

TABLE 1
Adhesion assay

| | Number of adherent cells (mean) | | |
|---|---|---|---|
| Time (minutes) | With ICDX | CONTROL | p |
| 0 | 0 | 0 | |
| 5 | 0 | 0 | |
| 10 | 0 | 0 | |
| 15 | 0 | 4 | 0.03 |
| 20 | 1 | 1.5 | 0.6 |
| 25 | 3 | 2.25 | 0.4 |
| 30 | 27.5 | 13 | 0.2 |
| 60 | 47.75 | 76 | 0.5 |
| 90 | 102 | 173.5 | 0.4 |
| 120 | 85 | 270.75 | 0.2 |

Cell Migration Assay.

In the CHEMO well the migration rate was significantly slower at H4 and H6 than in the CONTROL well (p=0.0496). The comparison of the other well at the different time didn't show a difference statistically significant (FIG. 2).

In Vivo Experimentation.

Twenty-four mice were injected, five in each group except for the toxicity group (n=4).

Analysis of Morbidity and Mortality

No death was related to PC or the treatment. We didn't notice any signs of dehydration or pain. No excessive loss or gain of weight were observed. Only one mouse died because of a liver injury during the IP injection. In the toxicity group, we didn't notice anything particular. The post mortem examination was normal.

Analysis of Peritoneal Carcinomatosis

PC grew in every mice. Mean PCI was 4,6+/−3,4 with 2,6+/−1,6 areas involved. Seven mice had ascites during the autopsy.

In the control group, the PCI was significantly higher than in the chemo group (p=0.02) and in the chemo ICDX group (p=0.03). There was no difference between the control group and the ICDX group (p=0.2).

TABLE 2
PCI in the differents groups.

| | | | | ICDX |
|---|---|---|---|---|
| | Control | ICDX | Chemo | Chemo |
| | group | group | group | group |
| | (n = 5) | (n = 4) | (n = 5) | (n = 5) |
| Mouse 1 | 11 | 5 | 5 | 3 |
| Mouse 2 | 7 | 9 | 4 | 1 |
| Mouse 3 | 4 | 5 | 2 | 1 |
| Mouse 4 | 10 | 4 | 3 | 1 |
| Mouse 5 | 10 | x | 2 | 1 |
| Mean PCI (SD) | 8.4 (2.9) | 5.75 (2.2) | 3.2 (1.3) | 1.4 (0.9) |

PCI in ICDX chemo group was significantly lower than in chemo group (p=0.04). PCI in ICDX chemo was also lower than in ICDX group (p=0.01).

TABLE 3
Number of involved areas in the differents groups.

| | | | | ICDX |
|---|---|---|---|---|
| | Control | ICDX | Chemo | Chemo |
| | group | group | group | group |
| | (n = 5) | (n = 4) | (n = 5) | (n = 5) |
| Mouse 1 | 5 | 2 | 4 | 3 |
| Mouse 2 | 3 | 5 | 3 | 1 |
| Mouse 3 | 2 | 2 | 1 | 1 |
| Mouse 4 | 5 | 2 | 2 | 1 |
| Mouse 5 | 5 | x | 2 | 1 |
| Mean (SD) | 4(1.4) | 2.75 (1.5) | 2.4 (1.1) | 1.4 (0.9) |

There was no difference in the different group concerning the number of involved areas expect when we compared the control group and the ICDX chemo group (p=0.02).

One mouse in the control group and one mouse in the chemo group had ascites. Three and two in the ICDX and ICDX chemo respectively had ascites. There were no differences between the groups.

Histological Analysis

This analysis showed a very poorly differentiated carcinoma. It was invasive and without functional architecture. There was a lot of mitoses. Cells had an hyperchromatic nuclei and a poor cytoplasm. Most of ICDX samples were partially necrosed but we couldn't determine if this necrosis was due to the treatment or a sign of particular agressivity of the tumor.

Bioluminescence Imaging.

This technology does not fit to compare the different group. Indeed the deep nodules, especially those which were on the mesentery or on the diaphragmatic cupolae, did not appear on the picture. Furthermore, if small nodules were hidden by bigger nodules, it was impossible to determine the number of nodules (data not shown). There were no correlations between PCI and bioluminescence pictures. The signals were also quickly saturated which didn't allow us to quantify the PC. The signals also really depend on the position of the mouse (data not shown).

This imaging was useful to verify if the graft worked. No mouse had at first a signal and then switch it off during the experiment.

However, this helped us to look for the smallest nodules in the small PC. Indeed, we took a picture after the laparotomy. At this time, there were a good correlation between macroscopic PCI and the pictures (data not shown).

### DISCUSSION

In vitro, ICDX has an effect on multiplication and adhesions of the tumoral cells.

In vivo, ICDX alone does not prevent implantation of tumoral cells in the peritoneum and the formation of the PC. However, when associated with intraperitoneal chemotherapy with 5-FU and oxaliplatin, ICDX seems to prevent PC formation or at least reduce it. ICDX has a synergetic effect with chemotherapy.

The use of mediator as carrier solutions for intraperitoneal chemotherapy has been described (22-27). With those therapy, the biodisponibility of the chemotherapy is higher. Many types of mediator were tried (gelatin microspheres, hyaluronic acid-based hydrogels . . . ) and different type of cancer were used (ovarian tumor, gastric tumor . . . ). In every case, the treatment was efficient on the PC without systemic toxicity. However, the PC was already settle down. Our model is original because it is the only model of high risk PC.

Limitations of our model can explain the inefficiency of isolated ICDX. This is a highly aggressive carcinoma undifferenciated. This phenotype is rarely present in human colonic cancer and is similar to a sarcomatic phenotype. As the expected effect of ICDX is low, it may have been hidden by the aggressivity of the tumor. Unfortunately, this is the only colonic murin cancer available and we thought important to work in a integral immune system to preserve the microenvironnement of the tumor. Moreover, on the contrary of the human peritoneum, presence of alpha-amylase in the murin peritoneum was described (28). Biodisponibility of ICDX may be lower in the murin peritoneum.

ICDX associated with intraperitoneal chemotherapy, deliver at the end of curative resection for a high risk of PC colonic cancer, seems to be an interessant therapeutic option.

However, two questions have to be answered. Indeed, we need to evaluate the safety of ICDX on digestive anastomosis. Results on animals are inconsistent. Many studies demonstrate that ICDX isn't associated to a higher risk of anastomotic leak (29-32). Pascual et al. have a rate of anastomotic leak of 10% (2/20) in ileocolic anastomosis in rabbits (31). A recent review does not describe a severe side effect due to ICDX on 764 patients without any specification about anastomotic leak (33). Catena et al., in a prospective randomised trial, didn't higlight an increase in anastomotic leak in small intestine anastomosis when ICDX is used (4% in each group). Hosie et al. report 5% of anastomotic leak in colonic anastomosis (1/22) which is conform with the rate in the litterature (34,35).

Eventually, ICDX has already being tested as a carrier solution for chemotherapy (36-38). In 2006, Hosie et al. run a pilot study for patients with colonic cancer Dukes' B or C without any metastasis. At the end of the resection of the primitive tumor, a catheter was put in the peritoneum and ICDX was used since day one, daily, with a rising quantity until 2 liters a day. Then 5FU was administred with ICDX twice a day during 15 days per month. Twenty two patients had the treatment. The follow-up found one recurrence at 12 months over the 10 patients which had the treatment. This study differ from our study because ICDX is used as an adjuvant therapy for all cancer without focusing on high risk PC patients. But this is interesting because this study showed no toxicity of repeated exposition to ICDX. Those studies allow us to think that ICDX behaves as a neutral substance to carry chemotherapy without modifying the pharmacocinetics or the pharmacodynamics of this solution.

This work developped a new model of growing PC in a murin model. This model is reliable and reproducible. PC was lower if chemotherapy and ICDX were injected simultaneously. This treatment is easy handling, suitable for a public or private hospital use. ICDX seems to be an element in the therapeutic arsenal of the treatment of high risk of PC colonic cancer. Morbidity of this solution needs to be evaluate in a prospective study on anastomotic leak.

### EXAMPLE 2

We have conducted a second study using the same experiment model to test the effect of Icodextrin and Oxaliplatin regarding an ovarian peritoneal carcinomatosis.

For that experiment, a human ovarian cancer cell line, named OVCAR is used, as prior exposed for the CT-26 cells. The only difference is the mice receiving cell injection, because OVACR is a human cancer cell, a nude mice animal is used because of immune deficiency of the animal.

Results at 21 days confirmed that Icodextrin decreases the carcinomatosis and association of Icodextrin and chemotherapy is well tolerated with no death animal.

The mean PCI (peritoneal cancer index) is at 7.25 for control group (no treatment), 4 for Icodextrin group, 3,7 for Oxaliplatin group and 3 for association Icodextrin and Oxaliplatin group (FIG. 5).

### REFERENCES

Throughout this application, various references describe the state of the art to which this invention pertains. The disclosures of these references are hereby incorporated by reference into the present disclosure.
1. Brodsky J T, Cohen A M. Peritoneal seeding following potentially curative resection of colonic carcinoma: implications for adjuvant therapy. Dis Colon Rectum. 1991 August; 34(8):723-7.
2. Elferink M A G, de Jong K P, Klaase J M, Siemerink E J, de Wilt J H W. Metachronous metastases from colorectal cancer: a population-based study in North-East Netherlands. Int J Colorectal Dis. 2015 February; 30(2):205-12.
3. Klaver C E L, Musters G D, Bemelman W A, Punt C J A, Verwaal V J, Dijkgraaf M G W, et al. Adjuvant hyperthermic intraperitoneal chemotherapy (HIPEC) in patients with colon cancer at high risk of peritoneal carcinomatosis; the COLOPEC randomized multicentre trial. BMC Cancer. 2015; 15:428.
4. Segelman J, Granath F, Holm T, Machado M, Mahteme H, Martling A. Incidence, prevalence and risk factors for peritoneal carcinomatosis from colorectal cancer. Br J Surg. 2012 May; 99(5):699-705.
5. Klaver Y L B, Lemmens V E P P, Nienhuijs S W, Luyer M D P, de Hingh I H J T. Peritoneal carcinomatosis of colorectal origin: Incidence, prognosis and treatment options. World J Gastroenterol W J G. 2012 Oct. 21; 18(39):5489-94.
6. Verwaal V J, Bruin S, Boot H, van Slooten G, van Tinteren H. 8-year follow-up of randomized trial: cytoreduction and hyperthermic intraperitoneal chemotherapy versus systemic chemotherapy in patients with peritoneal carcinomatosis of colorectal cancer. Ann Surg Oncol. 2008 September; 15(9):2426-32.
7. Elias D, Lefevre J H, Chevalier J, Brouquet A, Marchal F, Classe J-M, et al. Complete cytoreductive surgery plus intraperitoneal chemohyperthermia with oxaliplatin for peritoneal carcinomatosis of colorectal origin. J Clin Oncol Off J Am Soc Clin Oncol. 2009 Feb. 10; 27(5):681-5.
8. Elias D, Gilly F, Quenet F, Bereder J M, Sidéris L, Mansvelt B, et al. Pseudomyxoma peritonei: a French multicentric study of 301 patients treated with cytoreductive surgery and intraperitoneal chemotherapy. Eur J Surg Oncol J Eur Soc Surg Oncol Br Assoc Surg Oncol. 2010 May; 36(5):456-62.
9. Honore C, Goéré D, Souadka A, Dumont F, Elias D. Definition of patients presenting a high risk of developing peritoneal carcinomatosis after curative surgery for colorectal cancer: a systematic review. Ann Surg Oncol. 2013 January; 20(1):183-92.
10. Elias D, Honoré C, Dumont F, Ducreux M, Boige V, Malka D, et al. Results of systematic second-look surgery plus HIPEC in asymptomatic patients presenting a high risk of developing colorectal peritoneal carcinomatosis. Ann Surg. 2011 August; 254(2):289-93.
11. Joyce J A, Pollard J W. Microenvironmental regulation of metastasis. Nat Rev Cancer. 2009 April; 9(4):239-52.
12. Sandoval P, Jiménez-Heffernan J A, Rynne-Vidal Á, Pérez-Lozano M L, Gilsanz Á, Ruiz-Carpio V, et al. Carcinoma-associated fibroblasts derive from mesothelial cells via mesothelial-to-mesenchymal transition in peritoneal metastasis. J Pathol. 2013 December; 231(4):517-31.
13. Loureiro J, Aguilera A, Selgas R, Sandoval P, Albar-Vizcaíno P, Pérez-Lozano M L, et al. Blocking TGF-β1 protects the peritoneal membrane from dialysate-induced damage. J Am Soc Nephrol JASN. 2011 September; 22(9):1682-95.
14. Spaeth E L, Dembinski J L, Sasser A K, Watson K, Klopp A, Hall B, et al. Mesenchymal stem cell transition to tumor-associated fibroblasts contributes to fibrovascular network expansion and tumor progression. PloS One. 2009; 4(4):e4992.
15. Karnoub A E, Dash A B, Vo A P, Sullivan A, Brooks M W, Bell G W, et al. Mesenchymal stem cells within tumour stroma promote breast cancer metastasis. Nature. 2007 Oct. 4; 449(7162):557-63.
16. Rafii A, Mirshahi P, Poupot M, Faussat A-M, Simon A, Ducros E, et al. Oncologic trogocytosis of an original stromal cells induces chemoresistance of ovarian tumours. PloS One. 2008; 3(12):e3894.
17. Pasquet M, Golzio M, Mery E, Rafii A, Benabbou N, Mirshahi P, et al. Hospicells (ascites-derived stromal cells) promote tumorigenicity and angiogenesis. Int J Cancer J Int Cancer. 2010 May 1; 126(9):2090-101.
18. Catena F, Ansaloni L, Di Saverio S, Pinna A D, World Society of Emergency Surgery. P.O.P.A. study: prevention of postoperative abdominal adhesions by icodextrin 4% solution after laparotomy for adhesive small bowel obstruction. A prospective randomized controlled trial. J Gastrointest Surg Off J Soc Surg Aliment Tract. 2012 February; 16(2):382-8.
19. Trew G, Pistofidis G, Pados G, Lower A, Mettler L, Wallwiener D, et al. Gynaecological endoscopic evaluation of 4% icodextrin solution: a European, multicentre, double-blind, randomized study of the efficacy and safety in the reduction of de novo adhesions after laparoscopic gynaecological surgery. Hum Reprod Oxf Engl. 2011 August; 26(8):2015-27.
20. Klink C D, Schickhaus P, Binnebosel M, Jockenhoevel S, Rosch R, Tolba R, et al. Influence of 4% icodextrin solution on peritoneal tissue response and adhesion formation. BMC Surg. 2013; 13:34.
21. Otto J, Jansen P L, Lucas S, Schumpelick V, Jansen M. Reduction of peritoneal carcinomatosis by intraperitoneal administration of phospholipids in rats. BMC Cancer. 2007; 7:104.
22. Gunji S, Obama K, Matsui M, Tabata Y, Sakai Y. A novel drug delivery system of intraperitoneal chemotherapy for peritoneal carcinomatosis using gelatin microspheres incorporating cisplatin. Surgery. 2013 November; 154(5):991-9.
23. Liu L, Wu Q, Ma X, Xiong D, Gong C, Qian Z, et al. Camptothecine encapsulated composite drug delivery system for colorectal peritoneal carcinomatosis therapy: biodegradable microsphere in thermosensitive hydrogel. Colloids Surf B Biointerfaces. 2013 Jun. 1; 106:93-101.
24. Emoto S, Yamaguchi H, Kamei T, Ishigami H, Suhara T, Suzuki Y, et al. Intraperitoneal administration of cisplatin via an in situ cross-linkable hyaluronic acid-based hydrogel for peritoneal dissemination of gastric cancer. Surg Today. 2014 May; 44(5):919-26.
25. Oun R, Plumb J A, Wheate N J. A cisplatin slow-release hydrogel drug delivery system based on a formulation of the macrocycle cucurbit[7]uril, gelatin and polyvinyl alcohol. J Inorg Biochem. 2014 May; 134:100-5.
26. Bajaj G, Kim M R, Mohammed S I, Yeo Y. Hyaluronic acid-based hydrogel for regional delivery of paclitaxel to intraperitoneal tumors. J Control Release Off J Control Release Soc. 2012 Mar. 28; 158(3):386-92.
27. Picaud L, Thibault B, Mery E, Ouali M, Martinez A, Delord J-P, et al. Evaluation of the effects of hyaluronic acid-carboxymethyl cellulose barrier on ovarian tumor progression. J Ovarian Res. 2014; 7:40.
28. van den Tol P, Raa S ten, van Grevenstein H, Marquet R, van Eijck C, Jeekel H. Icodextrin reduces postoperative adhesion formation in rats without affecting peritoneal metastasis. Surgery. 2005 March; 137(3):348-54.
29. Baca B, Boler D E, Onur E, Akca O, Hamzaoglu I, Karahasanoglu T, et al. Icodextrin and Seprafilm do not interfere with colonic anastomosis in rats. Eur Surg Res Eur Chir Forsch Rech Chir Eur. 2007; 39(5):318-23.
30. Rodgers K E, Verco S J S, diZerega G S. Effects of intraperitoneal 4% icodextrin solution on the healing of bowel anastomoses and laparotomy incisions in rabbits. Colorectal Dis Off J Assoc Coloproctology G B Irel. 2003 July; 5(4):324-30.
31. Pascual I, Fernández de Miguel G, García Arranz M, García-Olmo D. Biosutures improve healing of experimental weak colonic anastomoses. Int J Colorectal Dis. 2010 December; 25(12):1447-51.
32. Ko [symbol] O, Dağ A, Ocal A K, Dirlik M M, Cömelekoğlu U, Gümü [symbol] L T, et al. [Effects of abdominal adhesion-preventing 4% icodextrin solution on healing of bowel anastomoses]. Ulus Travma Ve Acil Cerrahi Derg Turk J Trauma Emerg Surg TJTES. 2013 July; 19(4):305-12.
33. Broek R P G ten, Stommel M W J, Strik C, van Laarhoven C J H M, Keus F, van Goor H. Benefits and harms of adhesion barriers for abdominal surgery: a systematic review and meta-analysis. Lancet Lond Engl. 2014 Jan. 4; 383(9911):48-59.
34. Bakker I S, Grossmann I, Henneman D, Havenga K, Wiggers T. Risk factors for anastomotic leakage and leak-related mortality after colonic cancer surgery in a nationwide audit. Br J Surg. 2014 March; 101(4):424-32; discussion 432.
35. Krarup P-M, Jorgensen L N, Andreasen A H, Harling H, Danish Colorectal Cancer Group. A nationwide study on anastomotic leakage after colonic cancer surgery. Colorectal Dis Off J Assoc Coloproctology G B Irel. 2012 October; 14(10):e661-7.
36. Hosie K B, Kerr D J, Gilbert J A, Downes M, Lakin G, Pemberton G, et al. A pilot study of adjuvant intraperitoneal 5-fluorouracil using 4% icodextrin as a novel carrier solution. Eur J Surg Oncol J Eur Soc Surg Oncol Br Assoc Surg Oncol. 2003 April; 29(3):254-60.
37. McArdle C S, Kerr D J, O'Gorman P, Wotherspoon H A, Warren H, Watson D, et al. Pharmacokinetic study of 5-fluorouracil in a novel dialysate solution: a long-term intraperitoneal treatment approach for advanced colorectal carcinoma. Br J Cancer. 1994 October; 70(4):762-6.
38. Kerr D J, Young A M, Neoptolemos J P, Sherman M, Van-Geene P, Stanley A, et al. Prolonged intraperitoneal infusion of 5-fluorouracil using a novel carrier solution. Br J Cancer. 1996 December; 74(12):2032-5.

1. A method for the prophylactic treatment of peritoneal carcinomatosis in a patient in need thereof comprising administering to the patient a therapeutically effective combination of at least one dextrin polysaccharide and at least one chemotherapeutic agent.

2. The method of claim 1 wherein the peritoneal carcinomatosis results from a primary or a secondary carcinoma.

3. The method of claim 1 wherein the peritoneal carcinomatosis results from a malignant mesothelioma, benign papillary mesothelioma, desmoplastic small round cell tumors, peritoneal angiosarcoma, leiomyomatosis peritonealis disseminata (LPD), or a peritoneal hemangiomatosis

4. The method of claim 1 wherein the peritoneal carcinomatosis results from an ovarian cancer arising in women after bilateral oophorectomy.

5. The method of claim 1 wherein the peritoneal carcinomatosis results from a cancer of the endometrium, fallopian tubes, ovaries, uterus, colon, rectum, small bowel, gall bladder, bile duct, appendix, stomach, pancreas, liver or breast.

6. The method of claim 1 wherein the peritoneal carcinomatosis results from results from an ovarian cancer.

7. A method of reducing the risk of peritoneal carcinomatosis in a patient that has had an intra-abdominal cancer removed, said method comprising administering to said patient a therapeutically effective combination of at least one dextrin polysaccharide and at least one chemotherapeutic agent.

8. The method of claim 7 wherein the intra-abdominal cancer is located at or near the colon, at or near the ovary, at or near the rectum, at or near the stomach, or at or near the pancreas of the patient.

9. The method of claim 7 wherein the patient suffers from a metastatic cancer.

10. The method of claim 1 wherein the dextin polysaccharide is icodextrin.

11. The method of claim 1 wherein the chemotherapeutic agent is selected from the group consisting of an alkylating agent; an alkyl sulfonate; an aziridine; an ethylenimine or a methylamelamine; a nitrogen mustard a nitrosurea; an antibiotic; an anti-metabolite; a folic acid analogue; a purine analog; a pyrimidine analog; an androgen; an anti-adrenal; frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′2″-trichlorotriethylamine; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; chlorambucil; gemcitabine; 6-thioguanine; mercaptopurine; methotrexate; a platinum analog; etoposide (VP-16); ifosfamide; mitomycin C; mitoxantrone; vinblastine; vincristine; vinorelbine; navelbine; novantrone; teniposide; daunomycin; aminopterin; xeloda; ibandronate; difluoromethylornithine (DMFO); retinoic acid; an esperamicin; capecitabine; imexon; a tyrosine kinase inhibitor; and pharmaceutically acceptable salts, acids or derivatives of any of the above.

12. The method of claim 1 wherein the chemotherapeutic agent is capecitabine, carboplatin, cisplatin, cyclophosphamide, docetaxel, doxorubicin, epirubicin, erlotinib, 5-fluorouracil, gemcitabine, irinotecan, leucovorin, oxaliplatin, paclitaxel or topotecan.

13. The method of claim 1 wherein the dextrin polysaccharide and the chemotherapeutic agent are administered to the patient in the same pharmaceutical composition.

14. A pharmaceutical composition comprising at least one dextrin polysaccharide and at least one chemotherapeutic agent.

15. The method of claim 11, wherein
the alkylating agent is thiotepa or cyclosphosphamide;
the alkyl sulfonate is busulfan, improsulfan or piposulfan;
the aziridine is benzodopa, carboquone, meturedopa, or uredopa;
the ethylenimine or methylamelamine is altretamine, triethylenemelamine,
trietylenephosphoramide, triethylenethiophosphaoramide or trimethylolomelamine;
the nitrogen mustard is chlorambucil, chlornaphazine, cholophosphamide, estramustine, ifosfamide, mechlorethamine, mechlorethamine oxide hydrochloride, melphalan, novembichin, phenesterine, prednimustine, trofosfamide or uracil mustard;
the nitrosurea is carmustine, chlorozotocin, fotemustine, lomustine, nimustine or ranimustine.
the antibiotic is aclacinomycin, actinomycin, authramycin, azascrine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin, mycophenolic acid, nogalamycin, olivomycin, peplomycin, porfiromycin, puromycin, quelamycin, rodorubicin, streptonigrin, streptozocin, tubercidin, ubenimex, zinostatin or zorubici;
the anti-metabolite is methotrexate or 5-fluorouracil (5-FU);
the folic acid analogue is denopterin, methotrexate, pteropterin or trimetrexate;
the purine analog is fludarabine, 6-mercaptopurine, thiamiprine or thioguanine;
the pyrimidine analog is ancitabine, azacitidine, 6-azauridine, carmofur, cytarabine, dideoxyuridine, doxifluridine, enocitabine or floxuridine;
the androgen is calusterone, dromostanolone propionate, epitiostanol, mepitiostane or testolactone;
the anti-adrenal is aminoglutethimide, mitotane or trilostane;
the platinum analog is cisplatin or carboplatin; and/or
the tyrosine kinase inhibitor is epidermal growth factor receptor tyrosine kinase inhibitor erlotinib.
